(19) United States
(12) Patent Application Publication
(10) Pub. No.: US 2026/0283494 A1
(43) Pub. Date: Sep. 24, 2026

(54) SYSTEMS AND METHODS FOR CALCULATING VOLTAGES TO APPLY TO WOUND TISSUE

(71) Applicant: Solventum Intellectual Properties Company, Maplewood, MN (US)

(72) Inventors: Stephen M. KENNEDY, Maplewood, MN (US); Chaodi LI, Maplewood, MN (US); Zhijie ZHU, Maplewood, MN (US); Alexandra L. CRAMPTON, Maplewood, MN (US); Elias WILKEN-RESMAN, Maplewood, MN (US); Lindsey L. HINES, Maplewood, MN (US); Done DEMIRGOZ, Maplewood, MN (US); Howard M. KAPLAN, Maplewood, MN (US); Kevin T. REDDY, Maplewood, MN (US); Jeffrey G. ZINN, Maplewood, MN (US)

(73) Assignee: Solventum Intellectual Properties Company, Maplewood, MN (US)

(21) Appl. No.: 19/475,676

(22) PCT Filed: May 8, 2024
(86) PCT No.: PCT/US2024/028240

§ 371 Date: Oct. 15, 2025

Related U.S. Application Data
(60) Provisional application No. 63/500,989, filed on May 9, 2023.

(51) Int. Cl. A61B 5/0536; A61B 5/00

(52) U.S. Cl. CPC A61B 5/0536; A61B 5/445

(57) ABSTRACT

The present disclosure provides a method. The method includes applying one or more electrical signals to a wound tissue and collecting electrical measurements from the array of electrodes. The method also includes processing the collected electrical measurements to generate one or more impedance maps of the wound tissue that represent inhomogeneous electrical properties measured over an area of the wound tissue. The method further includes calculating at least two voltages, each targeted to the wound tissue, based on a spatial distribution of the electrical measurements of the impedance map(s) of the wound tissue. A system is also provided, including an array of electrodes, circuitry functionally connected to the array of electrodes, and a processor. Additionally, a device to apply to a wound tissue is provided, including an array of electrodes configured to be disposed on the wound tissue, and circuitry functionally connected to the array of electrodes.

### BACKGROUND

Epithelial cells can be directed to move in the direction of an applied electric field. This phenomenon can be exploited to enhance the re-epithelialization of wounds if electrical stimulation can be administered in an effective manner. Further developments in electrical stimulation for re-epithelialization would be desirable.

### SUMMARY

In a first aspect, a method is provided. The method comprises applying, via an array of electrodes, one or more electrical signals to a wound tissue; and collecting, via a circuitry functionally connected to the array of electrodes, electrical measurements from the array of electrodes. The method further comprises processing, via a processor, the collected electrical measurements to generate one or more impedance maps of the wound tissue that represent inhomogeneous electrical properties measured over an area of the wound tissue; and calculating at least a first voltage and a second voltage, each targeted to the wound tissue, based on a spatial distribution of the electrical measurements of the one or more impedance maps of the wound tissue.

In a second aspect, a system is provided. The system comprises an array of electrodes configured to apply one or more electrical signals to a wound tissue; circuitry functionally connected to the array of electrodes to collect electrical measurements therefrom; and a processor. The processor is configured to: process the collected electrical measurements to generate one or more impedance maps of the wound tissue that represent inhomogeneous electrical properties measured over an area of the wound tissue; and calculate at least a first voltage and a second voltage, each targeted to the wound tissue, based on a spatial distribution of the electrical measurements of the one or more impedance maps of the wound tissue.

In a third aspect, a device to apply to a wound tissue is provided. The device comprises an array of electrodes comprising at least two electrodes configured to be disposed on the wound tissue and configured to apply one or more electrical signals to the wound tissue; and circuitry functionally connected to the array of electrodes to collect electrical measurements therefrom and transfer the collected electrical measurements for processing.

These aspects advantageously provide calculated voltages tailored to specific wound tissue based on impedance maps of the wound tissue.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a flow diagram of an exemplary method, according to one embodiment.

FIG. 2 is a schematic diagram depicting an exemplary array of electrodes, according to one embodiment.

FIGS. 3A-3B are photos and maps related to estimating a wound boundary, according to one embodiment.

FIGS. 4A-4C are schematic diagrams depicting applying an electric field to wound tissue, according to one embodiment.

FIG. 5 depicts how an inwardly directed E-field may be achieved near a wound boundary of a wound tissue, according to one embodiment.

FIG. 6 is a schematic diagram depicting an exemplary system, according to one embodiment.

FIGS. 7A-7B are photographs of wound tissues on a pig's back and EIT-acquired maps of wound tissue conductivity, according to one embodiment.

FIG. 7C is a schematic diagram illustrating a system applicable to the wound tissues of FIGS. 7A-7B, according to one embodiment.

FIG. 8 is a flow diagram of one embodiment of the present disclosure.

FIGS. 9A-9C are illustrations of relative conductivity and topographical maps during progression of a wound, according to one embodiment.

While the above-identified figures set forth several embodiments of the disclosure other embodiments are also contemplated, as noted in the description. The figures are not necessarily drawn to scale. In all cases, this disclosure presents aspects of the invention by way of representation and not limitation.

### DETAILED DESCRIPTION

### Glossary

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

As used herein, the term “impedance” refers to an electrical property which is a complex quantity including what are referred to as “real” and “imaginary” quantities, e.g., Z=R+iX, where Z is the impedance, R is the so-called real component and is the resistance, and X is the so-called imaginary component and is the reactance. Additionally, the term “conductivity” is used here which is the mathematical inverse of the resistance R. The term “relative conductivity” refers to the conductivity relative to some baseline value, either measured or algorithmically estimated.

The term “electrical measurement(s)” refers to measurement(s) of electrical properties such as conductivity, resistivity, complex impedance, impedance magnitude, admittance, impedance phase angle, reactance, etc., at one or more frequencies.

The term “impedance map(s)” refers to representation(s) of spatial distributions of one or more electrical measurements, which may present in the form of map(s) or other suitable data structures.

The term “wound tissue” refers to both periwound tissue and wound bed tissue. The term “wound bed tissue” refers to tissue having damage to epithelial layers, damage to subcutaneous tissue, and/or tissue having at least one of a bruise, a rash, or an infection. The term “periwound tissue” refers to tissue in the periwound area, which can be defined as the area of skin extending to a certain distance (e.g., several centimeters, such as 4 cm) beyond the wound bed, or the surrounding skin extending from the wound bed.

The term “boundary” with respect to wound tissue refers to at least one of an area, a volume, or a line between a wound bed and periwound tissue. Wound tissue may have more than one enclosed boundary.

The term “tissue characteristics map(s)” or “clinical metrics” refers to representation(s) of spatial distributions of one or more of tissue characteristics including, for example, wound edges/boundaries, wound depth information (e.g., a topographical map of a wound with depth versus x and y coordinates), presence of granulation tissue, granulation tissue thickness, wound healing stage (e.g., hemostasis, inflammation, proliferation, remodeling stage, etc.), epithelial coverage, epithelial layer thickness, biomass, bioburden, infection level, infection type, necrotic tissue, healed tissue, etc. A tissue characteristics map may present in the form of map(s) or other suitable data structures. In some cases, a tissue characteristics representation or map may include a healing metrics.

The term “healing metrics” refers to global assessment(s) of a wound that represent calculations performed on tissue characteristic data. A healing metrics may include wound length, wound width, wound depth (e.g., maximum, minimum, average, etc.), wound area, wound volume, granulation tissue thickness (e.g., maximum, minimum, average, etc.), total epithelial coverage (e.g., percent of a wound bed covered in new epithelium), epithelial thickness (e.g., maximum, minimum, average, etc.), total bioburden, biofilm thickness (e.g., maximum, minimum, average, etc.), biofilm amount, etc.

### Methods

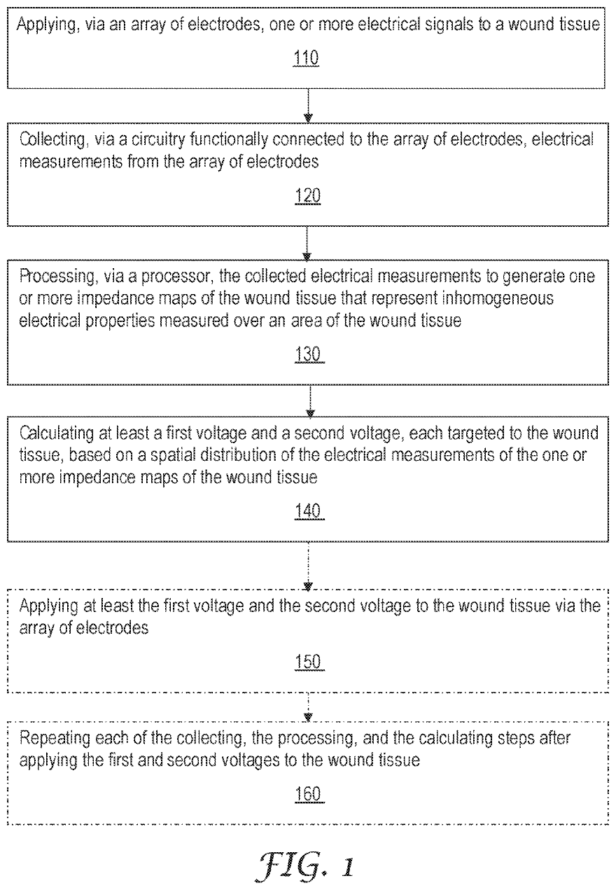

In a first aspect, a method is provided. Referring to FIG. 1, the method comprises:
applying, via an array of electrodes, one or more electrical signals to a wound tissue 110; collecting, via a circuitry functionally connected to the array of electrodes, electrical measurements from the array of electrodes 120;
processing, via a processor, the collected electrical measurements to generate one or more impedance maps of the wound tissue that represent inhomogeneous electrical properties measured over an area of the wound tissue 130; and
calculating at least a first voltage and a second voltage, each targeted to the wound tissue, based on a spatial distribution of the electrical measurements of the one or more impedance maps of the wound tissue 140. In some cases, the first voltage and the second voltage are different from each other, while in other cases the first voltage and the second voltage are the same. In select embodiments, at least four voltages are calculated, each targeted to the wound tissue, based on the spatial distribution of the electrical measurements of the one or more impedance maps of the wound tissue.

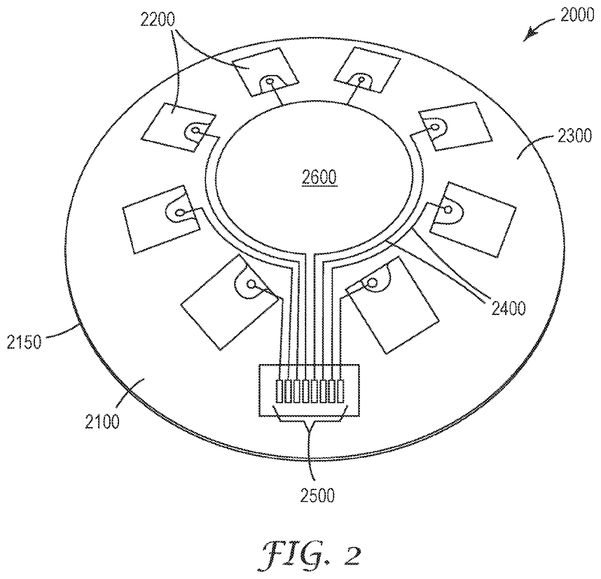

The electrodes are not particularly limited. Exemplary suitable electrode arrays include for instance and without limitation, 15 pin electrodes on a rigid printed circuit board (PCB), 15 pin electrodes on flexible Kapton PCB, 64 pad electrodes on flexible Kapton PCB, 16 pin electrodes on flexible clear composite, or 3M RED DOT 2670 skin electrodes attached to flexible Kapton PCB via snap-connectors. Referring to FIG. 2, an exemplary 8-electrode device 2000 was made on a soft and flexible substrate 2100. The electrodes 2200 were 3M RED DOT 2360 electrodes (3M Company, St. Paul, MN). The device included a UV-curable silicone encapsulant protective layer 2300, and a urethane film 2150 which was laser-etched and bladed with silver to serve as electrical leads 2400 and contacts 2500, with the electrodes 2200 embedded in the cured silicone encapsulant 2300. The substrate 2100 defines an open area 2600 that may be suitably positioned over a desired portion of wound tissue.

Electrical impedance tomography (EIT) is used for measuring and determining spatial distributions of electrical impedance within a continuous two-dimensional (2D) or three-dimensional (3D) space. Typically, impedance measurements are obtained from electrical contacts sparsely distributed in the continuous 2D/3D space, and an impedance map of that continuous 2D/3D is reconstructed by solving the inverse problem of a Finite Element Model (FEM) to space. For methods described herein, an array of electrodes is placed on wound tissue in order to spatially map a resistive/conductive profile of the wound and nearby tissues. In some cases, one or more of the electrodes is placed about the perimeter of the wound bed on the periwound tissue, one or more of the electrodes is placed on the wound bed, or both.

The electronics to implement EIT mapping of the wound tissue may include the electrodes, a microcontroller for measurement of control and data acquisition, a low noise precision current source as a power supply, an Analog-to-Digital (ADC) preamplifier for noise filtering and signal amplification, and input/output multiplexers for switching of electrodes for current sourcing and voltage measurements.

One or more signal generators can be electrically connected to the array of electrodes and configured to generate an alternating electrical signal, e.g., an electrical waveform. The electrical signal may be sinusoidal, a square wave, a pulse wave, a triangle wave, a sawtooth wave, and the like. The signal generator may be configured to generate an electrical signal including one or more frequencies at any frequency, including between 1 kHz to 2 kHz, between 2 kHz to 4 kHz, between 4 kHz to 55 kHz, between 55 kHz to 120 kHz. In some examples, the signal generator may be configured to generate an electrical signal in a frequency range that may be greater than or less than the example ranges above. In some examples, the electrical signal generator may be configured to generate an electrical signal at a predetermined frequency, such as approximately 85 kHz (e.g., 85 kHz+10 kHz). In some cases, the signal generator is configured to generate electrical signals.

The impedance maps may be obtained at a single frequency or multiple frequencies. In some embodiments, an impedance may be measured with respect to different baselines established through different estimation schemes. The baseline may refer to a map of conductivity values that represent the tissue before wounding. A relative conductivity may refer to the current conductivity map of the tissue subtracted from a baseline map.

In some examples, a baseline measurement of an unwounded tissue may include a homogeneous measurement which captures the background conductivity of the intact tissue, and an inhomogeneous measurement which captures the conductivity of wound tissue. Methods for estimation of baseline may include, for example, a frequency-difference EIT (fdEIT), measurement-scale features (MSF), best homogeneous (BH) estimators, data-driven estimators, or a combination thereof. Data-driven estimators can include machine learning and deep causal learning methods that use database references. Useful database references can be based on patient history or patient demographics. As an alternative, it is also possible to reconstruct a conductivity map of wounded tissue using a method that does not require a baseline measurement of unwounded tissue. Because biological tissues can have distinctive frequency responses, impedance distribution of the wound tissue can be imaged by a fdEIT reconstruction method, in which measurements are taken under at least two frequencies on wounded tissue. Measurement at the first frequency (i.e., reference frequency) can function as a proxy for a baseline measurement, while measurement at the second frequency (i.e., measuring frequency) serves as an inhomogeneous measurement. As such, in some embodiments, the one or more impedance maps of the wound tissue are algorithmically estimated using frequency-difference electrical impedance tomography (fdEIT), measurement-scale features (MSF), best homogeneous (BH) estimators, data-driven estimators, or a combination thereof.

Different methods can be used in reconstructing the conductivity maps. Exemplary methods include a one-step Gauss-Newton (GN) method and an iterative Total Variation (TV) method, in each case applying a suitable hyperparameter. Hyperparameters can be determined heuristically to optimize the degree of contrast between the anomaly and background. Note this heuristic selection can also be replaced with automatic selection based on any pre-determined strategy. The one-step GN method is capable of providing real-time reconstruction results with acceptable quality of shape and size of the anomalies. The TV method, being an iterative method, tends to be computationally slower compared to the GN method, but can also afford a higher resolution of topological features.

As a further alternative, baseline measurement estimation techniques can be applied to compute a homogeneous conductivity distribution of the unwounded tissue. TdEIT can then take the estimated baseline measurement and the inhomogeneous measurement to reconstruct the conductivity map of the wound tissue. For instance, either a best homogeneous approximation or pre-defined MSFs can be used to estimate a baseline measurement. In a EIT system with n_{E} electrodes, the baseline measurement U_{baseline} represents a vector of n_{E} (n_{E}−3) voltage measurements under the setting of adjacent stimulation pattern.

In one embodiment, U_{baseline} is estimated by the following steps. First, a finite element model (FEM) that reflects the geometry of the wound tissue and electrode arrangement is generated. Second, a simulated baseline measurement U₀ is obtained from the FEM with a homogeneous conductivity distribution, where baseline conductivity σ₀=1. Third, an inhomogeneous measurement Uₘₑₐₛ is acquired from the wound tissue. Finally, U₀ is scaled by a ratio parameter to estimate the baseline measurement. The baseline vector would thus be expressed as:

$${\tilde{U}}_{baseline} = μ {U}_{0}$$

When using BH method for baseline estimation, the ratio parameter y can be expressed as

$$μ = {\sum }_{i = 1}^{{n}_{E}} {\sum }_{j = 1}^{{n}_{E} - 3} [ {( {U}_{m e a s} )}_{i , j} \cdot {( {U}_{0} )}_{i , j} ] / {\sum }_{i = 1}^{{n}_{E}} {\sum }_{j = 1}^{{n}_{E} - 3} {[ {( {U}_{0} )}_{i , j} ]}^{2}$$

When using MSF operators for baseline estimation, the ratio parameter y can be expressed as

$$μ = \frac{f ( {U}_{meas} )}{f ( {U}_{0} )}$$

where f is defined as the feature operator that maps measurements to a feature value. Exemplary MSF operators include an arithmetic mean, range, midrange, electrode-based average range, and electrode-based average midrange. Mathematical representations for each are provided below:

### MSF1: Arithmetic Mean

$${f}_{m e a n} ( U ) = \frac{1}{{n}_{E} ( {n}_{E} - 3 )} {\sum }_{i = 1}^{{n}_{E}} {\sum }_{j = 1}^{{n}_{E} - 3} {U}_{i , j}$$

### MSF2: Range

$${f}_{r a n g e} ( U ) = max ( U ) - min ( U )$$

### MSF3: Midrange

$${f}_{m i d r a n g e} ( U ) = \frac{1}{2} ( max ( U ) - min ( U ) )$$

### MSF4: Electrode-Based Average Range

$${f}_{average} ( U ) = \frac{1}{{n}_{E}} {\sum }_{i = 1}^{{n}_{E}} ( max \{ {U}_{{n}_{E} , 1} , {U}_{{n}_{E} , 2} , … , {U}_{{n}_{E} , ( {n}_{E} - 3 )} \} - min \{ {U}_{{n}_{E} , 1} , {U}_{{n}_{E} , 2} , … , {U}_{{n}_{E} , ( {n}_{E} - 3 )} \} )$$

### MSF5: Electrode-Based Average Midrange

$${f}_{avemidrange} ( U ) = \frac{1}{2 {n}_{E}} {\sum }_{i = 1}^{{n}_{E}} ( max \{ {U}_{{n}_{E} , 1} , {U}_{{n}_{E} , 2} , … , {U}_{{n}_{E} , ( {n}_{E} - 3 )} \} - min \{ {U}_{{n}_{E} , 1} , {U}_{{n}_{E} , 2} , … , {U}_{{n}_{E} , ( {n}_{E} - 3 )} \} )$$

In some embodiments, new impedance maps may be arithmetically calculated from impedance maps at given frequencies and from maps generated from different baseline estimation schemes. The measured impedance may vary because of variation in the electrical characteristics from tissue site to tissue site of an individual, e.g., different tissue locations on the same patient and/or animal, variation of tissue characteristics at different times, and variation from individual to individual, e.g., from patient to patient and/or animal to animal. For example, electrical characteristics of tissue may vary based on tissue composition and thickness, tissue water content and/or tissue hydration, ambient relative humidity at the time of measurement, and the like. In addition, electrical characteristics of tissues may depend on what specific tissue types are resident at that location. For example, variations are observed for muscle versus fat-laden tissues, and for the case of wound tissues, unwounded, well-epithelialized tissues versus open-wound areas with various types and amounts of healed tissues in them (e.g., different amounts of granular tissue filing in the wound bed and/or different degrees of epithelial coverage atop the wound bed).

In some embodiments, a map of wound tissue impedances can be translated to quantitative metrics of healing. In other words, spatial maps of impedances acquired via EIT (e.g., conductance or resistance) can be translated to quantitative maps of healing such as, for example, wound shape, depth, size (e.g., area or volume), amount of granular tissue, epithelial coverage, wound stage, etc.

In some embodiments, one or more impedance maps can be converted to spatial maps of clinical metrics by calibrating the impedance maps using a calibration model. The clinical metrics can include various wound information data versus (x, y) coordinates in a Cartesian coordinate system (x, y, z) where the z axis corresponds to the depth direction of the wound tissue. Example clinical metrics may include wound depth d (x, y), granular tissue thickness t_{g} (x, y), epithelial coverage c (x, y), biofilm thickness t_{b}(x, y), bioburden (i.e., the number of contaminated organisms found in a given amount of material) b (x,y), infection level i (x,y), healing stage (e.g., inflammation, proliferation, or remodeling stages) indication h (x,y), etc. Suitable calibration methods also include those described in detail in PCT Application No. PCT/IB2022/062138 (Zhu et al.), incorporated herein by reference in its entirety.

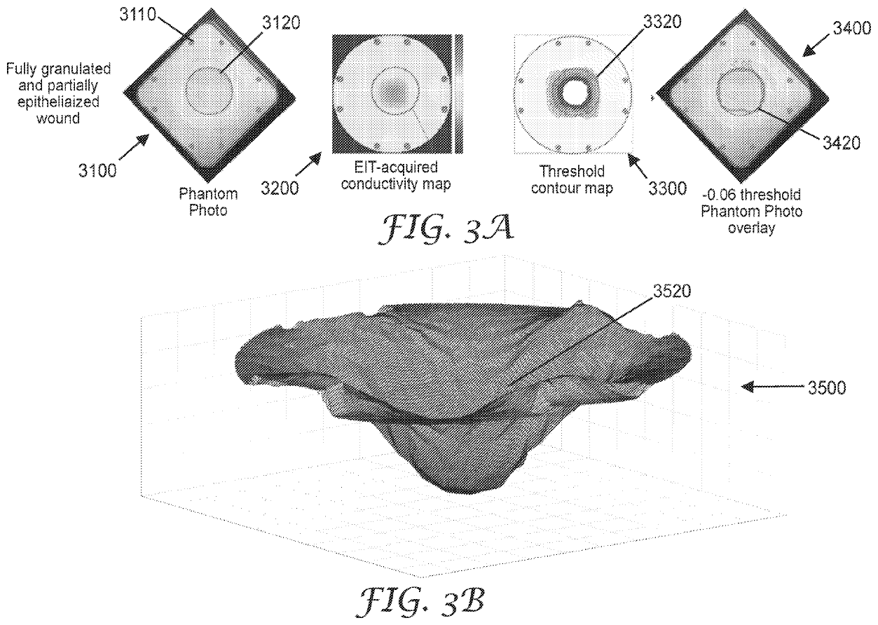

In some embodiments, the obtained impedance maps can be used to determine the wound boundaries and other wound tissue features. For example, in certain embodiments, the one or more impedance maps of the wound tissue identify at least one boundary between the wound bed and periwound tissue. As noted above, a “boundary” refers to at least one of an area, a volume, or a line between a wound bed and periwound tissue. For example, referring to FIG. 3, impedance map(s) of wound site relative conductivities can be used to estimate the location of the wound boundaries through thresholding. FIG. 3A shows a photo 3100 of tissue mimicking wound phantom modeling of a wound that has started to re-epithelialize from the wound boundary inward, which could possibly benefit from E-field-accelerated epithelialization. Dots 3110 indicate the locations of electrode placements and the circle 3120 indicates the location of the wound boundary (in this case, a circumscription that encloses non-epithelialized open wound tissue and excludes epithelized tissues). An EIT-acquired map 3200 of the wound phantoms' relative conductivity is also provided. FIG. 3A further includes a contour map 3300 of the wound phantom's relative conductivity with thresholds at −0.05, −0.06, −0.08, −0.10, −0.12, and −0.15. Note that the −0.06 threshold contour follows the wound boundary 3320 relatively well. A photo 3400 of the wound phantom with the −0.06 relative conductivity threshold highlights how this threshold predicts the location of the wound boundary 3420 relatively well. FIG. 3B depicts a threshold contour map 3500 with the threshold at −0.06 3520 as a 3D contour. A variety of mathematical methods can be applied here for wound edge detections based on the changes of material properties (relative conductivity, etc.). There are many methodologies (such as Canny edge detector, Deriche method, Sobel Method, Prewitt method, Laplacian of Gaussian, Determinant of Hessian, etc.) for edge detection available in literature, usually categorized in to two groups: search-based and zero-crossing based. For the case here one way is to apply the search-based methods, for example Canny edge detector or Sobel Method, based on the gradient magnitude of conductivity and search the gradient direction to find the wound edge. Another way is to apply the zero-crossing methods for searching zero crossings in a second-order derivative expression (for example, Laplacian). Or we can combine the edge detection methods, such as combination of evaluating the conductivity and its gradient, to determine the wound boundary.

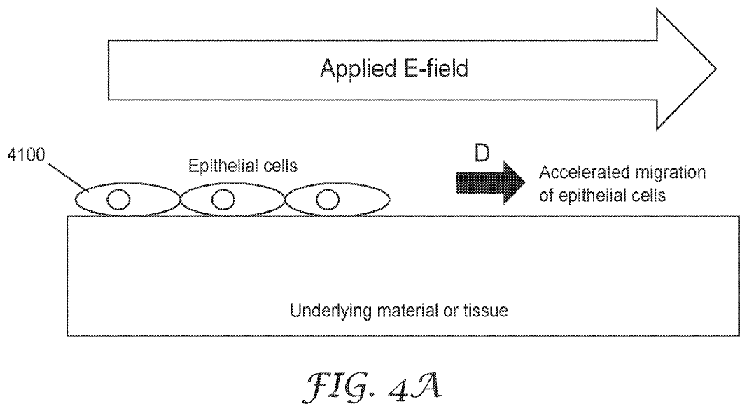

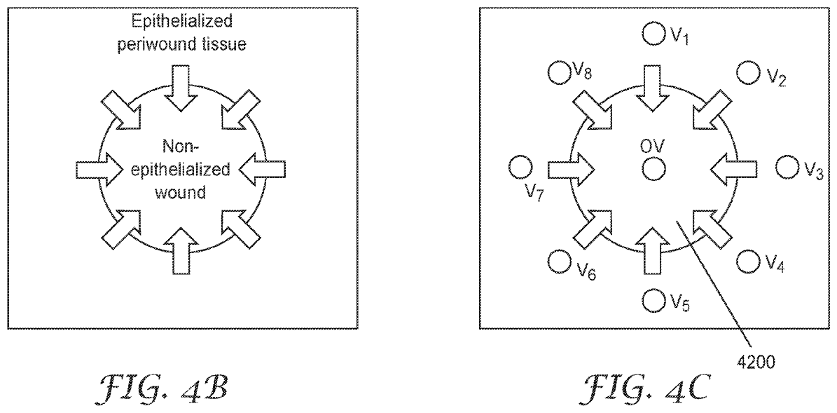

Referring, for instance, to FIG. 4, epithelial cells 4100 can be directed to move in the direction D of an applied electric field (e.g., FIG. 4A). This phenomenon can be exploited to enhance the re-epithelialization of wounds if electrical stimulation can be administered in a manner that forms an electric field (E-field) at a targeted therapeutic intensity (e.g., FIG. 4B). While an array of electrodes, providing voltages v1-v8, interfacing the wound tissue 4200 could theoretically generate a sculpted E-field profile (e.g., an inwardly directed E-field profile as depicted in FIG. 4C), the electrical properties of an actual wound tend to be spatially inhomogeneous and change over time (e.g., as the wound heals).

Advantageously, methods according to at least certain embodiments of the present disclosure produce calculated voltages that are designed to achieve a predetermined electric field targeted to wound tissue based on a near-real time determination of impedance maps of the wound tissue. As the electrical properties of a certain wound tissue change over time, the calculations can concomitantly be updated over time. Accordingly, referring back to FIG. 1, the method further optionally comprises repeating each of the collecting, the processing, and the calculating steps after applying the first and second voltages to the wound tissue 160. Additionally, a ground (i.e., 0 voltage) may also be applied to the wound tissue. In some cases, the collecting, the processing, and the calculating steps are repeated at a predetermined time after applying the first and second voltages to the wound tissue. Typically, these processes can be performed on a short timescale such as in seconds (or even more quickly), thus the predetermined time may be selected based on other criteria (e.g., an amount of time required for a measurable change to one or more electrical properties of the wound tissue). The predetermined time is not particularly limited, and may comprise any one or more of 1-60 minutes, 1-24 hours, 1-7 days, or 1-52 weeks.

One method for calculating voltages is as described herein. Voltage optimization calculations can be performed to select appropriate voltages at specified electrodes placed about the wound tissue (e.g., in locations 3110 in FIG. 3A). Note that for these examples, in order to streamline calculations, the two electrodes in the 12 o'clock position are tied to the same voltage (v₁), the two electrodes at 3 o'clock are tied to voltage (v₂), at 6 o'clock (v₃), at 9 o'clock (v₄), and the center electrode is tied to ground (0 V). Note, however, that the described methods can instead apply 8 independent voltages to each of these 8 electrodes and can use any number of electrodes and applied voltages. Voltages are calculated using the mapping of the wound tissue's electrical inhomogeneity (e.g., from EIT mapping) such that an E-field profile is formed in the tissue with a targeted 30 V/m intensity about the wound boundary, which is also mapped using EIT methods. In some embodiments, the E-field profile is designed to provide an inwardly directed E-field direction.

This method utilizes the principle of superposition. That is, the voltage distribution that manifests on the wound tissue is a sum of the voltage distributions formed from each single electrode's voltage contribution. The algorithm applies a voltage (vᵢ) to each individual electrode (i.e., i may be equal to [1, 2, 3, 4]). Finite Element simulations performed in COMSL's AC/DC module are applied to find the wound boundary voltage distributions and negative gradients:

$$Electric fields , \vec{E} = - ∇ V ,$$

which must be separated into x- and y-components. Hence:

$$\vec{{E}_{x}} = - \frac{\partial {V}_{i}}{\partial x} and \vec{{E}_{y}} = - \frac{\partial {V}_{i}}{\partial y}$$

for the i^{th} electrode where i=[1, 2, 3, 4].
Taking advantage of superposition, the E-fields are then:

$$\vec{{E}_{x}} = - {\sum }_{i = 1}^{4} {A}_{i} \frac{\partial {V}_{i}}{\partial x} and \vec{{E}_{y}} = - {\sum }_{i = 1}^{4} {A}_{i} \frac{\partial {V}_{i}}{\partial y} ,$$

where Aᵢ are scale factors for each individual electrode, which can be algorithmically weighted.
The voltage gradient norms are then calculated as:

$$VGN = \sqrt{{( - \frac{\partial V}{\partial x} )}^{2} + {( - \frac{\partial V}{\partial y} )}^{2}}$$

Optimization programs based on MATLAB's Optimization Function are used to solve for these weighted scalar factors Aᵢ where the error e is:

$$e = \sum {( VGN - {VG}_{critical} )}^{2} where$$

$${VG}_{critical} is the target E - field intensity , in our examples : 30 V / m .$$

Note that this target E-field intensity can be any value determined to optimize the migration of epithelial cells and can be informed by experimentation, databases, algorithms, machine leaning, deep causal learning and doctor/patient inputs (patient demographic, medical record, etc.). The optimal applied electrode voltages are then determined to be scaled by:
Aᵢ Vᵢ for electrodes i=[1, 2, 3, 4].

Referring again to FIG. 1, the method optionally further comprises applying at least the first voltage and the second voltage to the wound tissue via the array of electrodes 150. An array of electrodes includes a minimum of two electrodes, but any number can be used. For instance, an array of electrodes may include 3, 4, 5, 6, 7, 8, 9, 10, 11, 12, or more electrodes. Regardless of how many electrodes are present in an array, the electrodes may each be configured to act independently and/or to have any voltage, positive or negative.

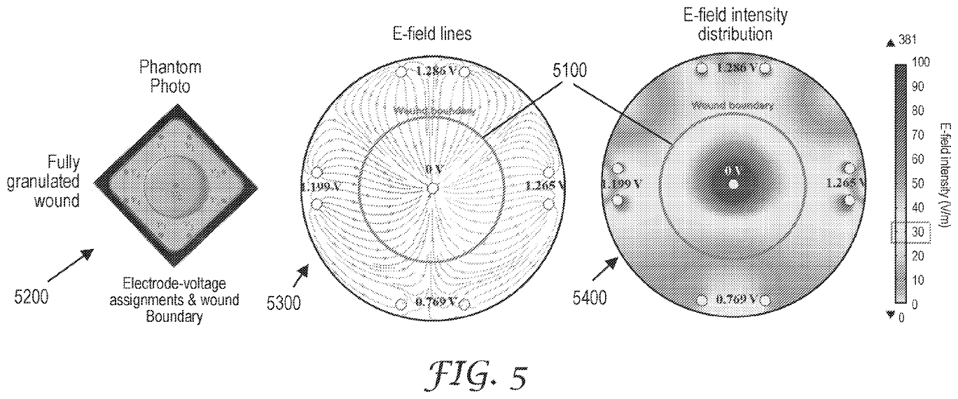

In some cases, applying the at least first and second voltages generates an electric field oriented at least partially inward from the boundary into the wound bed. For example, referring to FIG. 5, an inwardly directed E-field may be achieved near a wound boundary of a particular wound tissue. A photo 5200 is of a wound phantom showing the locations of each electrode v1-v8 and the grounded v0. A diagram 5300 is of E-field lines when electrodes are excited at the noted optimized voltages. Note that the E-field lines (i.e., lines with arrows) generally point inward starting at least at the wound boundary 5100. A diagram 5400 depicts the E-field distribution when electrodes are excited at the optimized voltages. Note that the target E-field intensity of nearly 30 V/m is achieved along the wound boundary.

The magnitude of the electric field is not particularly limited, however. In some cases, applying the (e.g., at least first and second) voltages generates an electric field having a magnitude of 0 Volts per meter (V/m) or greater, 2 V/m, 5 V/m, 10 V/m, 15 V/m, 20 V/m, 25 V/m, 30 V/m, 35 V/m, 40 V/m, 45 V/m, 50 V/m, 55 V/m, 60 V/m, 65 V/m, 70 V/m, or 75 V/m or greater; and 1,000 V/m or less, 950 V/m, 900 V/m, 850 V/m, 800 V/m, 750 V/m, 700 V/m, 650 V/m, 600 V/m, 550 V/m, 500 V/m, 450 V/m, 400 V/m, 350 V/m, 300 V/m, 250 V/m, 200 V/m, 175 V/m, 150 V/m, 125 V/m, 100 V/m, 90 V/m, 85 V/m, 80 V/m, 75 V/m, 70 V/m, 65 V/m, 60 V/m, 55 V/m, or 50 V/m or less. In certain embodiments, an E-field intensity ranges from 10 V/m to 100 V/m, inclusive.

Referring again to FIGS. 4C and 5, in some embodiments, the applying comprises applying a 0 Volt (V) ground to an electrode that is positioned on the wound bed. In select embodiments, including the example shown in FIG. 5, an electrode that is positioned on the wound bed is located substantially at a center of the wound bed.

### Systems and Devices

In a second aspect, a system is provided. The system comprises:
an array of electrodes configured to apply one or more electrical signals to a wound tissue;
circuitry functionally connected to the array of electrodes to collect electrical measurements therefrom; and
a processor configured to:
   process the collected electrical measurements to generate one or more impedance maps of the wound tissue that represent inhomogeneous electrical properties measured over an area of the wound tissue; and
   calculate at least a first voltage and a second voltage, each targeted to the wound tissue, based on a spatial distribution of the electrical measurements of the one or more impedance maps of the wound tissue.

In a third aspect, a device to apply to a wound tissue is provided. The device comprises:
an array of electrodes comprising at least two electrodes configured to be disposed on the wound tissue and configured to apply one or more electrical signals to the wound tissue; and
circuitry functionally connected to the array of electrodes to collect electrical measurements therefrom and transfer the collected electrical measurements for processing.

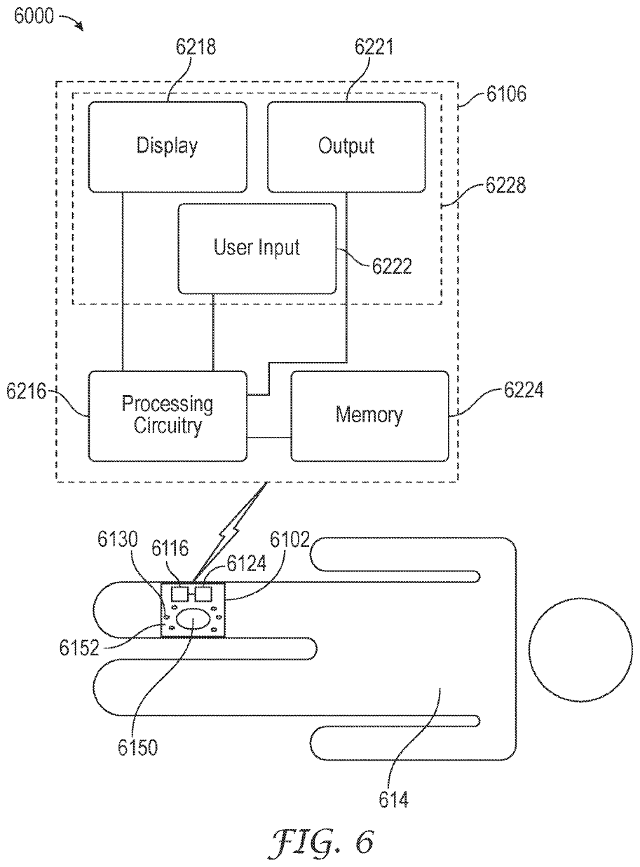

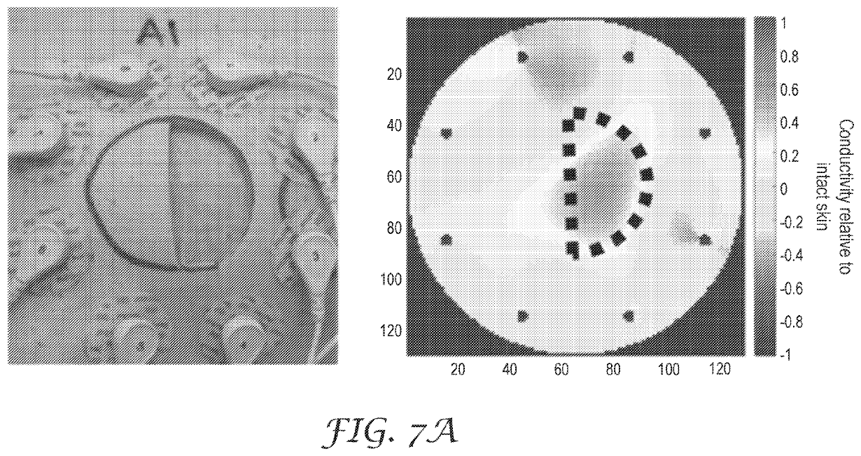

FIG. 6 is a schematic diagram depicting an exemplary system 6000, according to one embodiment. In the depicted embodiment, the system 6000 includes a device 6102 and a computing device 6106. In some cases, the device 6102 is a diagnostic or monitoring device. In some cases, the device 6102 is a dressing. The device 6102 may be communicatively coupled, for example by a wired or a wireless connection, to the computing device 6106. The computing device 6106 may include processing circuitry 6216 coupled to a display 6218, an output 6221, and a user input 6222 of a user interface 6228. In some examples, the display 6218 may include one or more display devices (e.g., monitor, PDA, mobile phone, tablet computer, any other suitable display device, or any combination thereof). For example, the display 6218 may be configured to display physiological information and information indicative of epithelial tissue characteristics determined by the system 6000.

The device 6102 may be any type of structure. In some examples, the device 6102 may include a bandage including a flexible backing material, an adhesive for bonding to the skin of patient 614, and electrodes 6130. In some examples, the device 6102 may include a foam dressing including electrodes 6130. In some examples, the device 6102 may include a material affixed to a tissue via, for example, adhesive, or being physically held in place. In other examples, the device 6102 may be a diagnostic patch, for example, a material including any of electrodes 6130. In some examples, additional materials may be applied to a patient 614 for wound measuring/monitoring, for example, sterile saline-laden gauze, a gel, or the like, placed between the device 6102 and the tissue site 6150.

The device 6102 includes an array of electrodes 6130. When the device 6102 is disposed on wound tissue to be tested, the array of electrodes can apply electrical signals from a signal generator to a first tissue site 6152 located outside a second tissue site 6150. The second tissue site 6150 may correspond to a wounded tissue or a wound bed, e.g., tissue having damage to epithelial layers and/or subcutaneous tissue. The second tissue site 6150 may also correspond to tissue having a bruise, tissue having a rash, tissue having an infection, and the like. The second tissue site 6150 may also correspond to currently undamaged tissue that need to be monitored for injury (e.g., to monitor for Venous Leg Ulcers (VLUs) or Pressure Ulcers (PUs)), in cases where there is an observable open wound. The first tissue site 6152 optionally corresponds to tissue in the periwound area, which can be defined as the area of skin extending to a certain distance (e.g., several centimeters such as 4 cm) beyond the wound bed, or the surrounding skin extending from the wound bed. In some examples, additional materials may include treatments such as medications and/or may be at least partially electrically conductive and may enhance electrical conductivity between the electrodes 6130 and the first tissue site 6152.

One or more signal generators can be electrically connected to the array of electrodes 6130 and configured to generate an alternating electrical signal, e.g., an electrical waveform. The electrical signal may be as described above in detail with respect to the first aspect.

In the depicted embodiment of FIG. 6, the device 6102 further includes a processing circuitry 6116 and a memory 6124. In some embodiments, the device 6102 may process the electrical signals without transferring the electrical signals to the computing device 6106. For example, the processing circuitry 6116 may further include a signal monitor to detect electrical signals applied to the first tissue site 6152 proximate to the second tissue site 6150. In other embodiments, the electrical signals, or information corresponding to the electrical signals, may be transferred to the computing device 6106 for processing, for example, by a wired or wireless connection between the device 6102 and the computing device 6106.

The memory 6116, as well as memory 6224, may include any volatile or non-volatile media, such as a random-access memory (RAM), read only memory (ROM), non-volatile RAM (NVRAM), electrically erasable programmable ROM (EEPROM), flash memory, and the like. A memory may be a storage device or other non-transitory medium. A memory may be used by the processing circuitry 6216 or 6124 to, for example, store fiducial information or initialization information corresponding to physiological monitoring, such as wound monitoring. In some examples, the processing circuitry 6216 or 6124 may store physiological measurements or previously received data from electrical signals in the memory for later retrieval. In some examples, processing circuitry may store determined values, such as information indicative of epithelial tissue characteristics, or any other calculated values, in a memory for later retrieval.

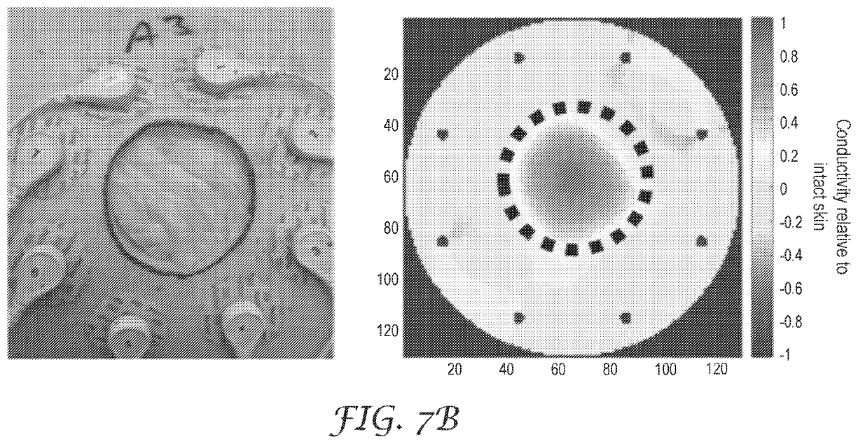

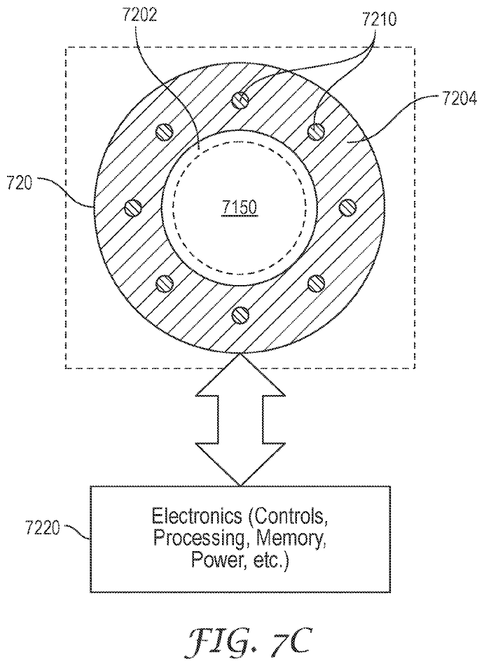

Each of FIGS. 7A and 7B is a photo of a tissue site with which a system may be employed, according to one embodiment. FIG. 7C is a schematic diagram illustrating the system 7000 applied to a wound tissue site, such as the wound tissue sites of FIGS. 7A-7B. As shown in the example of FIGS. 7A-7B, the tissue site 7150 corresponds to an at least partially open-wound tissue. A periwound tissue site 7152 corresponds to the area surrounding the open-wound tissue 7150.

In this example, the system 7000 includes an array of electrodes 7210 disposed on the periwound tissue site 7152. The array of electrodes 7210 is supported by a substrate 720. In the depicted example, the substrate 720 includes a central portion 7202 substantially covering the (e.g., open) wound bed tissue 7150 and a periphery 7204 of the central portion 7202. The array of electrodes 7210 is disposed on an inner surface of the dressing periphery 7204.

Utilizing the EIT methods described previously, the wound sites shown in FIGS. 7A and 7B can be mapped in manners that characterize the spatial distribution of the wound bed's electrical conductivity relative to the intact skin found in the wound site's periwound tissue. To collect these data, a single euthanized Yorkshire swine was prepped first by shaving the animal's dorsal right flank (where wound beds would be generated and periwound electrodes would be placed) and belly (where a single large reference electrode would be placed). Two 5-cm-diameter circular wound bed sites were outlined with a pen on the animal's dorsal right flank spaced 15 cm apart, wound center to center. One wound bed was generated via surgical excision at full-thickness (i.e., cut down to the dorsal fascia) with a semicircular shape (FIG. 7A, left) and the other wound bed was generated at full-thickness with a circular shape (FIG. 7B, left). A large adhesive return electrode was placed on the animal's shaved belly which was connected to the EIT electronics' electrical ground. Eight 3M Red Dot 2670 electrodes were cut into circular shapes and placed around each of the two wound beds on the intact periwound tissue (8 electrodes per wound) with even spacing about the wound's perimeter (FIGS. 7A and 7B, left photos). These electrodes were snap-connected to the EIT electronics' input terminals via electrocardiogram (ECG) leads. The EIT electronics injected sinusoidal currents to rotating pairs of electrodes at 40 kHz using a Keithly 6211 current source while taking voltage measurements from other electrodes in the array. This tomographic mapping scheme resulted in spatial maps of the wound tissue's conductivity which is presented here relative to the periwound tissue's intact skin (FIGS. 7A and 7B, right conductivity maps). It can be seen for the semicircular wound bed, that the generated relative conductivity map presents an area of altered conductivity values in the region of the map that corresponds to the half-circle wound bed (FIG. 7A, at right: non-zero colors in the dashed semicircular region). Likewise, for the circular wound bed, the generated relative conductivity map presents an area of altered conductivity values in the region of the map that corresponds to the circular wound bed (FIG. 7B, at right: non-zero colors in the dashed circular region). This example demonstrates the ability to capture an impedance map on animal tissues that could be subsequently used to generate a map of wound boundaries. The impedance map and wound boundary map could then be fed into algorithms that determine optimal voltages to be applied to the system's electrodes to generate a target therapeutic E-field intensity about the wound boundary.

It is to be understood that in some examples, additional electrodes can be placed on the wound tissue, such as on the open wound bed. In some examples, electrodes can be exclusively on the periwound tissue, or on both the wound bed tissue and the periwound tissue. In some examples, electrodes that reside in the periwound are desirable in that (i) it is less invasive due to not having to touch sensitive wound bed tissue and (ii) an electrical interface with intact periwound tissue is likely more stable than the wound bed tissue because it changes over time (e.g., as it heals).

In some examples, electrodes are provided for a 4-proble measurement, with 2 electrodes for current sourcing, and 2 electrodes for voltage measurement, the minimum number of electrodes is 4. In some examples, 8 or more electrodes are provided to obtain mapping results. A higher number of electrodes may enable higher resolution and accuracy.

It is to be understood that any suitable format of electrode devices can be used to enable placement of an electrode array in a predetermined pattern on a tissue site. In some examples, the electrode array can be placed on the perimeter of a dressing such as, for example, on the drape of a Negative Pressure Wound Therapy (NPWT) dressing. In some examples, the electrode array can be integrated into a non-dressing device. In one example, a flexible circuit substrate can be decorated with snap connectors to which multiple electrodes can be connected. In one example, the device may include a flexible Printed Circuit Board (PCB) with an inner constellation of metal pin electrodes that can interface with the wound bed or with multiple metal pin electrodes that interface with the periwound tissues, circumscribing the wound. Similar configurations can be implemented in a rigid PCB format as well. Additional suitable electrodes are those described above with respect to the first aspect. The electrodes can be placed in any location on the wound tissue, such as within the wound bed, outside of the wound bed, or both.

Referring again to FIG. 7C, the system 7000 further includes an electronics component 7220 electrically connected to the array of electrodes 7210. The electronics component 7220 may include various control circuitry, processors, memory, power, etc. For example, the electronics component 7220 may include one or more of the processing circuitry 6116, the memory 6124, the processing circuitry 6216, and the memory 6224 as shown in FIG. 6.

The electronics component 7220 is configured to apply, via the array of electrodes 7210, electrical signals to the tissue site, collect electrical measurements from the array of electrodes 7210, process the collected electrical measurements to generate one or more impedance maps of the wound bed (e.g., the open-wound tissue 7150, and the subdermal wound tissue 7154) that represent inhomogeneous electrical properties measured over an area of the wound tissue, and calculate at least a first voltage and a second voltage, each targeted to the wound tissue, based on a spatial distribution of the electrical measurements of the one or more impedance maps of the wound tissue. As mentioned above, the one or more impedance maps could also include a baseline map representing unwounded tissue.

In certain preferred embodiments, the circuitry is further functionally connected to the array of electrodes to pass voltage therethrough, e.g., to the wound tissue. In select embodiments, the array of electrodes comprises a first electrode that is configured to apply the first voltage to the wound tissue and a second electrode that is configured to apply the second voltage to the wound tissue. It is advantageous to have the option to use the same electrodes for both collecting electrical measurements and delivering a voltage to wound tissue as the overall device is simpler than if different sets of electrodes were required or if separate devices were needed to create the impedance maps and apply electrical stimulation to wound tissue.

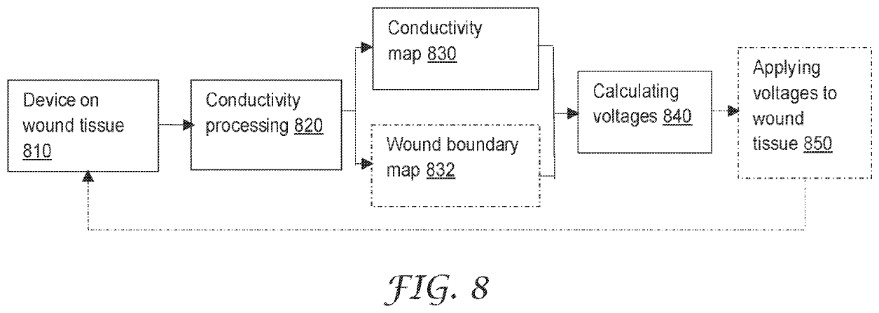

Referring to FIG. 8, a schematic diagram is provided depicting a flow chart of one embodiment according to the present disclosure. In such cases, a device (e.g., according to the third aspect) is placed on the wound tissue 810, then, based on collected electrical measurements from the device, conductivity processing 820 of the electrical measurements is performed. The processing generates a conductivity map 830 of the wound tissue and optionally also a wound boundary map 832, which may be employed in calculating voltages 840. Further, the embodiment optionally also includes applying the calculated voltages to the wound tissue 850. In some cases, advantageously, following applying the voltages to the wound tissue 850, the process is repeated to collect new electrical measurements from the device that is on the wound tissue 810 to capture differences in electrical measurements due to changes in conductivity of the wound tissue over time, e.g., from increased epithelialization as the wound tissue heals.

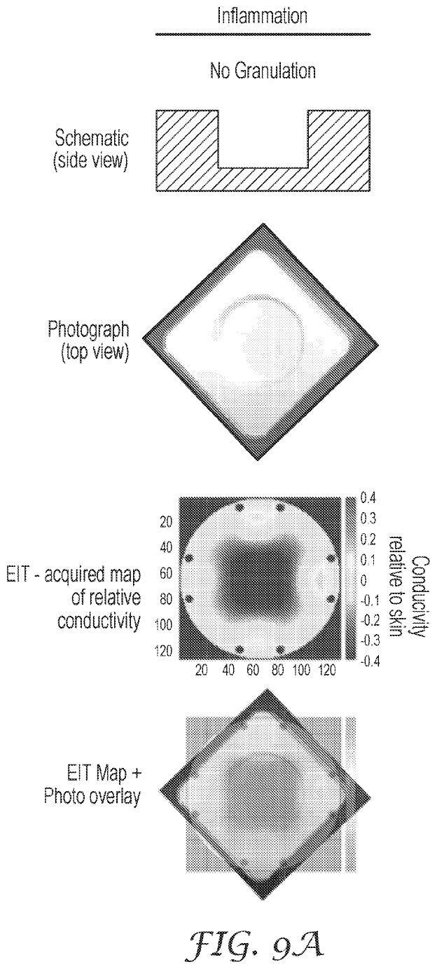

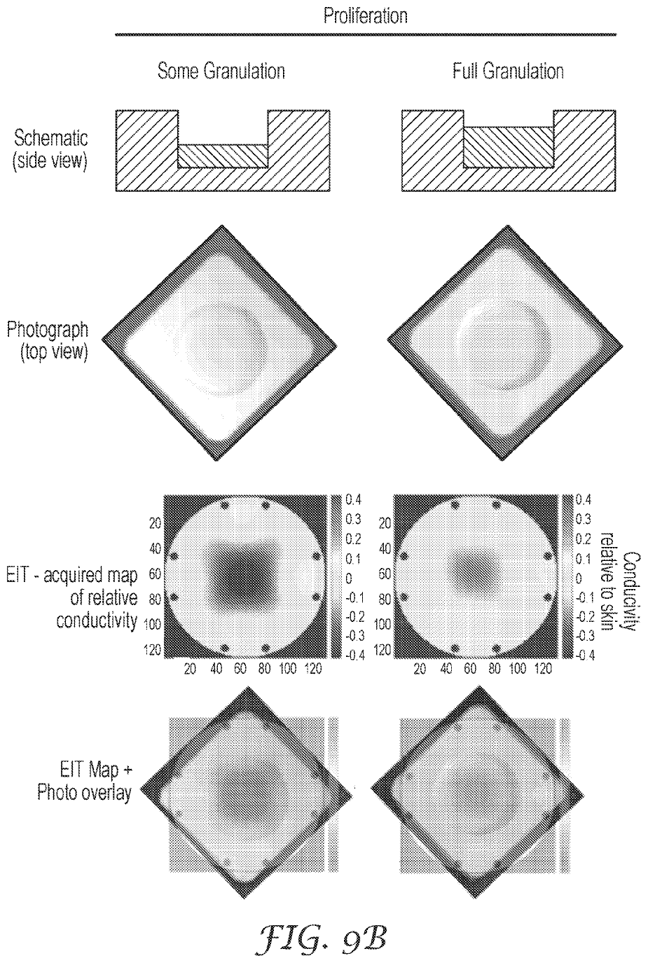

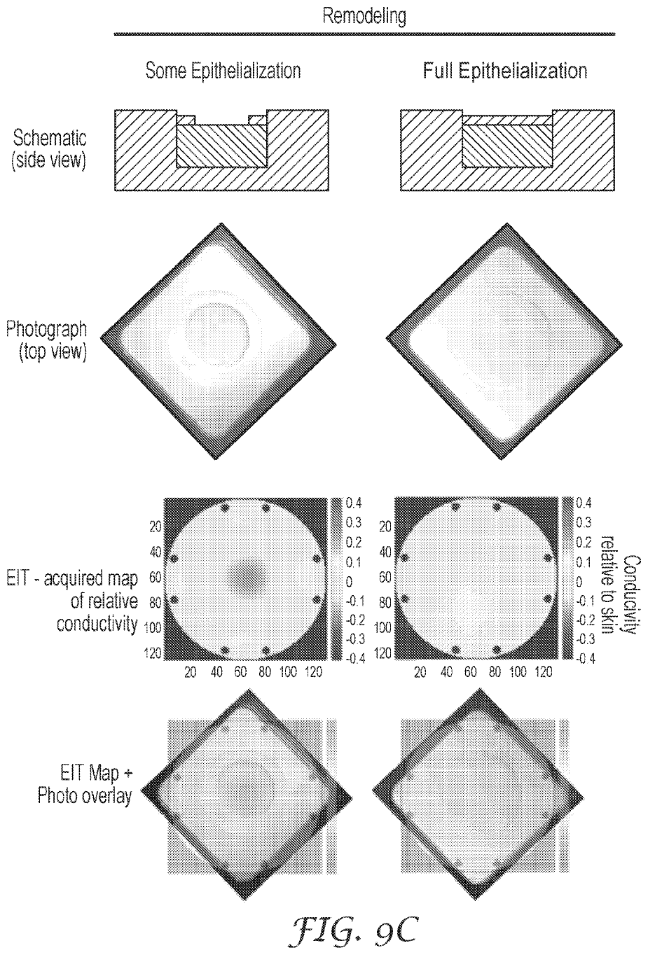

Referring to FIGS. 9A-9C, schematics are provided of tissue mimicking phantoms used in conductivity mapping experiments from a side view (top row). Each phantom represents a wound in progressively more healed states, moving from FIG. 9A to FIG. 9B to FIG. 9C. Top view photographs of the tissue mimicking wound phantoms are also provided (second row). Additionally, EIT-acquired maps of the wound phantoms' relative conductivities are shown in FIGS. 9A-9C (third row), plus overlays of the phantom photographs on the EIT maps (bottom row). In these cases, relative conductivity indicates differences in wound bed conductivity relative to that of periwound epithelialized skin (e.g., of periwound skin was 2×10⁶ S/m and considered to have a relative conductivity of 0; 0.2 relative conductivity is 20% greater than skin at 2.4×10⁶ S/m; and −0.2 relative conductivity is 20% less than skin at 1.6×10⁻⁶ S/m).

### EMBODIMENTS

In a first embodiment, the present disclosure provides a method. The method comprises applying, via an array of electrodes, one or more electrical signals to a wound tissue; and collecting, via a circuitry functionally connected to the array of electrodes, electrical measurements from the array of electrodes. The method further comprises processing, via a processor, the collected electrical measurements to generate one or more impedance maps of the wound tissue that represent inhomogeneous electrical properties measured over an area of the wound tissue; and calculating at least a first voltage and a second voltage, each targeted to the wound tissue, based on a spatial distribution of the electrical measurements of the one or more impedance maps of the wound tissue.

In a second embodiment, the present disclosure provides a method according to the first embodiment, wherein the first voltage and the second voltage are different from each other.

In a third embodiment, the present disclosure provides a method according to the first embodiment or the second embodiment, further comprising applying at least the first voltage and the second voltage to the wound tissue via the array of electrodes.

In a fourth embodiment, the present disclosure provides a method according to the third embodiment, wherein the applying comprises applying a 0 Volt (V) ground to an electrode that is positioned on the wound bed.

In a fifth embodiment, the present disclosure provides a method according to the fourth embodiment, wherein the electrode that is positioned on the wound bed is located at a center of the wound bed.

In a sixth embodiment, the present disclosure provides a method according to any of the second through fifth embodiments, further comprising repeating each of the collecting, the processing, and the calculating steps after applying the first and second voltages to the wound tissue.

In a seventh embodiment, the present disclosure provides a method according to the sixth embodiment, wherein the collecting, the processing, and the calculating steps are repeated at a predetermined time after applying the first and second voltages to the wound tissue.

In an eighth embodiment, the present disclosure provides a method according to any of the second through seventh embodiments, wherein the applying the at least first and second voltages generates an electric field having a magnitude of 0 to 1,000 Volts per meter (V/m).

In a ninth embodiment, the present disclosure provides a method according to any of the first through eighth embodiments, wherein the one or more impedance maps of the wound tissue identify at least one boundary between the wound bed and periwound tissue.

In a tenth embodiment, the present disclosure provides a method according to the ninth embodiment, wherein the applying the at least first and second voltages generates an electric field oriented at least partially inward from the boundary into the wound bed.

In an eleventh embodiment, the present disclosure provides a method according to any of the first through tenth embodiments, wherein at least four voltages are calculated, each targeted to the wound tissue, based on the spatial distribution of the electrical measurements of the one or more impedance maps of the wound tissue.

In a twelfth embodiment, the present disclosure provides a method according to any of the first through eleventh embodiments, wherein the one or more impedance maps of the wound tissue are algorithmically estimated and do not require a baseline measurement of unwounded tissue, wherein the one or more impedance maps of the wound tissue are algorithmically estimated using frequency-difference electrical impedance tomography (fdEIT), measurement-scale features (MSF), best homogeneous (BH) estimators, data-driven estimators, or a combination thereof.

In a thirteenth embodiment, the present disclosure provides a system. The system comprises an array of electrodes configured to apply one or more electrical signals to a wound tissue; circuitry functionally connected to the array of electrodes to collect electrical measurements therefrom; and a processor. The processor is configured to: process the collected electrical measurements to generate one or more impedance maps of the wound tissue that represent inhomogeneous electrical properties measured over an area of the wound tissue; and calculate at least a first voltage and a second voltage, each targeted to the wound tissue, based on a spatial distribution of the electrical measurements of the one or more impedance maps of the wound tissue.

In a fourteenth embodiment, the present disclosure provides a system according to the thirteenth embodiment, wherein the array of electrodes comprises a first electrode that is configured to apply the first voltage to the wound tissue and a second electrode that is configured to apply the second voltage to the wound tissue.

In a fifteenth embodiment, the present disclosure provides a device to apply to a wound tissue. The device comprises an array of electrodes comprising at least two electrodes configured to be disposed on the wound tissue and configured to apply one or more electrical signals to the wound tissue; and circuitry functionally connected to the array of electrodes to collect electrical measurements therefrom and transfer the collected electrical measurements for processing.

In a sixteenth embodiment, the present disclosure provides a device according to the fifteenth embodiment, wherein the circuitry is further functionally connected to the array of electrodes to pass voltage therethrough.

Although the present invention has been described with reference to preferred embodiments, workers skilled in the art will recognize that changes may be made in form and detail without departing from the spirit and scope of the invention.

1. A method comprising:
applying, via an array of electrodes, one or more electrical signals to a wound tissue;
collecting, via a circuitry functionally connected to the array of electrodes, electrical measurements from the array of electrodes;
processing, via a processor, the collected electrical measurements to generate one or more impedance maps of the wound tissue that represent inhomogeneous electrical properties measured over an area of the wound tissue; and
calculating at least a first voltage and a second voltage, each targeted to the wound tissue, based on a spatial distribution of the electrical measurements of the one or more impedance maps of the wound tissue.

2. The method of claim 1, wherein the first voltage and the second voltage are different from each other.

3. The method of claim 1, further comprising applying at least the first voltage and the second voltage to the wound tissue via the array of electrodes.

4. The method of claim 3, wherein the applying comprises applying a 0 Volt (V) ground to an electrode that is positioned on the wound bed.

5. The method of claim 4, wherein the electrode that is positioned on the wound bed is located at a center of the wound bed.

6. The method of claim 2, further comprising repeating each of the collecting, the processing, and the calculating steps after applying the first and second voltages to the wound tissue.

7. The method of claim 6, wherein the collecting, the processing, and the calculating steps are repeated at a predetermined time after applying the first and second voltages to the wound tissue.

8. The method of claim 2, wherein the applying the at least first and second voltages generates an electric field having a magnitude of 0 to 1,000 Volts per meter (V/m).

9. The method of claim 2, wherein the one or more impedance maps of the wound tissue identify at least one boundary between the wound bed and periwound tissue.

10. The method of claim 9, wherein the applying the at least first and second voltages generates an electric field oriented at least partially inward from the boundary into the wound bed.

11. The method of claim 1, wherein at least four voltages are calculated, each targeted to the wound tissue, based on the spatial distribution of the electrical measurements of the one or more impedance maps of the wound tissue.

12. The method of claim 1, wherein the one or more impedance maps of the wound tissue are algorithmically estimated and do not require a baseline measurement of unwounded tissue, wherein the one or more impedance maps of the wound tissue are algorithmically estimated using frequency-difference electrical impedance tomography (fdEIT), measurement-scale features (MSF), best homogeneous (BH) estimators, data-driven estimators, or a combination thereof.

13. A system comprising:
an array of electrodes configured to apply one or more electrical signals to a wound tissue;
circuitry functionally connected to the array of electrodes to collect electrical measurements therefrom; and
a processor configured to:
process the collected electrical measurements to generate one or more impedance maps of the wound tissue that represent inhomogeneous electrical properties measured over an area of the wound tissue; and
calculate at least a first voltage and a second voltage, each targeted to the wound tissue, based on a spatial distribution of the electrical measurements of the one or more impedance maps of the wound tissue.

14. The system of claim 13, wherein the array of electrodes comprises a first electrode that is configured to apply the first voltage to the wound tissue and a second electrode that is configured to apply the second voltage to the wound tissue.

15. A device to apply to a wound tissue comprising:
an array of electrodes comprising at least two electrodes configured to be disposed on the wound tissue and configured to apply one or more electrical signals to the wound tissue; and
circuitry functionally connected to the array of electrodes to collect electrical measurements therefrom and transfer the collected electrical measurements for processing.

16. The device of claim 15, wherein the circuitry is further functionally connected to the array of electrodes to pass voltage therethrough.
